(19) United States
(12) Patent Application Publication
(10) Pub. No.: US 2026/0325880 A1
(43) Pub. Date: Oct. 22, 2026

(54) RADIATION TREATMENT SYSTEM, AND CONTROL DEVICE AND CONTROL METHOD FOR RADIATION TREATMENT SYSTEM

(71) Applicant: HITACHI HIGH-TECH CORPORATION, Tokyo (JP)

(72) Inventors: Etsuji KATONO, Tokyo (JP); Motoki MATSUNO, Tokyo (JP); Toru UMEKAWA, Tokyo (JP)

(21) Appl. No.: 19/491,039

(22) PCT Filed: Jan. 29, 2024
(86) PCT No.: PCT/JP2024/002684

§ 371 Date: Dec. 8, 2025

(30) Foreign Application Priority Data
Jun. 14, 2023 (JP) 2023-097765

(51) Int. Cl. A61N 5/10; G06F 3/0483

(52) U.S. Cl. CPC A61N 5/1048; A61N 5/1045; A61N 2005/1074; G06F 3/0483

(57) ABSTRACT

A control device for a radiation therapy system in which steps of treatment transition in a workflow format includes a storage unit, a processing unit, and an interface including one or more display areas. The storage unit stores correspondence information between an image to be displayed in the display area when a transition is made to a predetermined step and the predetermined step. The processing unit switches an image in the display area based on the correspondence information when a transition is made to the predetermined step. The processing unit is capable of displaying an image different from an image associated with the predetermined step in the display area based on a designation operation from an operator. The interface has a function of receiving the designation operation and a function of receiving an operation for returning to display of the image associated with the predetermined step.

### TECHNICAL FIELD

The present invention relates to a radiation therapy system, a control device and a control method for the radiation therapy system.

### BACKGROUND ART

In the related art, JP2015-083068A (PTL 1) discloses a technique for controlling radiation therapy. This publication discloses that “a radiation therapy system 1 schematically includes a radiation irradiation system 10, a treatment planning device 101, an image server 102, a three-dimensional tomographic image capturing device 103, a positioning System 104, and the like. The radiation irradiation system 10 schematically includes a radiation generation device 12 for generating radiation, a transfer system 13 for transferring the radiation generated by the radiation generation device 12 to an irradiation device 14, the irradiation device 14 including an irradiation head (irradiation nozzle) for emitting the radiation, a gantry, and the like, a control unit 11 for controlling the radiation generation device 12, the transfer system 13, the irradiation device 14, and the like, a bed 15 for a patient to lie thereon, a bed driving device 105, and the like.”

### CITATION LIST

### Patent Literature

PTL 1: JP2015-083068A

### SUMMARY OF INVENTION

### Technical Problem

In the related art described above, there are a screen of the treatment planning device and a screen of the positioning system. In addition, a screen for displaying an image of an irradiation area acquired by an electronic portal image capturing device (EPID), an image obtained by imaging a state of a multi-leaf collimator (MLC), or c the like may be further provided. Since the radiation therapy system includes various devices, if each device has a display screen, the number of screens may be too large, causing a decrease in operability.

Therefore, an object of the present invention is to improve operability of a radiation therapy system.

### Solution to Problem

In order to achieve the above object, a representative control device for a radiation therapy system according to the invention is a control device for a radiation therapy system in which steps of treatment transition in a workflow format, the control device including a storage unit, a processing unit, and an interface including one or more display areas. The storage unit stores correspondence information between an image to be displayed in the display area when a transition is made to a predetermined step and the predetermined step. The processing unit switches an image in the display area based on the correspondence information when a transition is made to the predetermined step. The processing unit is capable of displaying an image different from the image associated with the predetermined step in the display area, based on a designation operation from an operator. The interface has a function of receiving the designation operation and a function of receiving an operation for returning to display of the image associated with the predetermined step.

A representative control method for a radiation therapy system according to the invention is a control method for a radiation therapy system in which steps of treatment transition in a workflow format, the control method to be executed by a control device including a storage unit, a processing unit, and an interface including one or more display areas, the control method including: storing, by the storage unit, correspondence information between an image to be displayed in the display area when a transition is made to a predetermined step and the predetermined step; switching, by the processing unit, an image in the display area based on the correspondence information when a transition is made to the predetermined step; displaying, by the processing unit, an image different from the image associated with the predetermined step in the display area, based on a first operation from an operator received by the interface; and returning, by the processing unit, to display of the image associated with the predetermined step, based on a second operation from the operator received by the interface.

### Advantageous Effects of Invention

According to the present invention, the operability of the radiation therapy system can be improved. Problems, configurations, and effects other than those described above will become apparent by the following description of embodiments.

### BRIEF DESCRIPTION OF DRAWINGS

FIG. 1 is a diagram illustrating screen display according to an embodiment.

FIG. 2 is a diagram illustrating a control device according to an embodiment.

FIG. 3 is a flowchart for illustrating a processing operation of the control device.

FIG. 4 is a flowchart illustrating display control in a step.

FIG. 5 is a diagram illustrating a screen of a display operation unit.

### DESCRIPTION OF EMBODIMENTS

Hereinafter, embodiments £ will be described with reference to the drawings.

### Embodiment 1

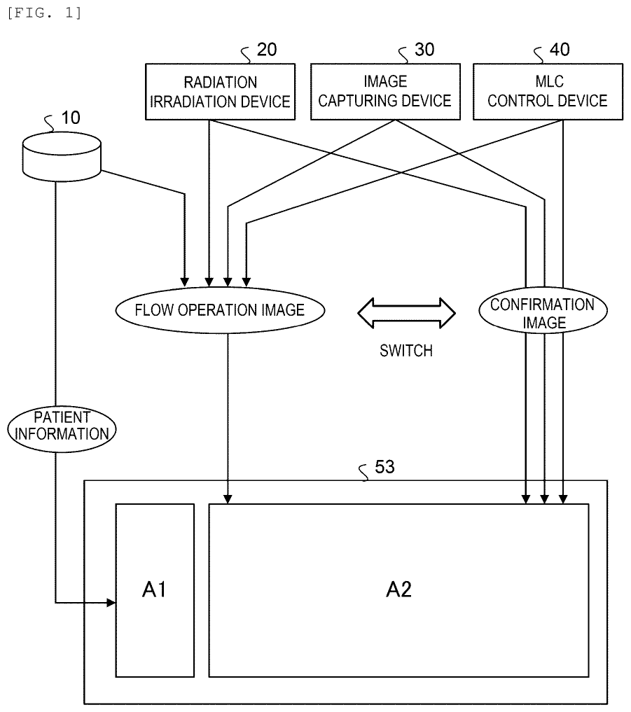

FIG. 1 is a diagram illustrating screen display according to an embodiment. A display operation unit 53 is, for example, a touch panel display, and is an example of an interface. The display operation unit 53 includes a display area A1 and a display area A2. The display operation unit 53 displays patient information acquired from a database 10 in the display area A1. The patient information is, for example, a name of a person to be subjected to radiation therapy and the like. In addition, the display operation unit 53 can acquire a treatment plan of the patient from the database 10 and display the treatment plan in the display area A1. A state of a couch or a gantry can also be displayed in the display area A1.

The display operation unit 53 displays a flow operation image in the display area A2. The flow operation image indicates information necessary for each step in the radiation therapy system in which steps of treatment transition in the workflow format, and an operation for transitioning to the next step is received. The flow operation image displays a basic image when an operator performs processing in each step, and for example, in a positioning step described later, displays, side by side, a patient image acquired at the time of therapy planning and a patient image acquired immediately before treatment. In this way, it is common that the images displayed to the operator are basically different depending on the step. The flow operation image may include a plurality of images, and provides an appropriate image in each step to the operator. Although details will be described later, the operator can sequentially advance a step of acquiring patient information from the database 10 and displaying the patient information, a step of acquiring an image related to the patient by an image capturing device 30 and confirming a position of the patient, a step of performing irradiation with radiation by a radiation irradiation device 20 while controlling an MLC by an MLC control device 40, and the like, mainly with reference to the flow operation image.

Further, the display operation unit 53 can switch the image displayed in the display area A2 to a confirmation image. The confirmation image is, for example, an image generated from an output of the radiation irradiation device 20, the image capturing device 30, or the MLC control device 40. The display operation unit 53 can receive an operation for returning to the flow operation image after displaying the confirmation image based on designation of the operator.

That is, the operator can advance the treatment step with the flow operation image, and can appropriately switch to a confirmation image of a desired device when necessary to confirm a state of the device, and then return to the flow operation image to advance the treatment step.

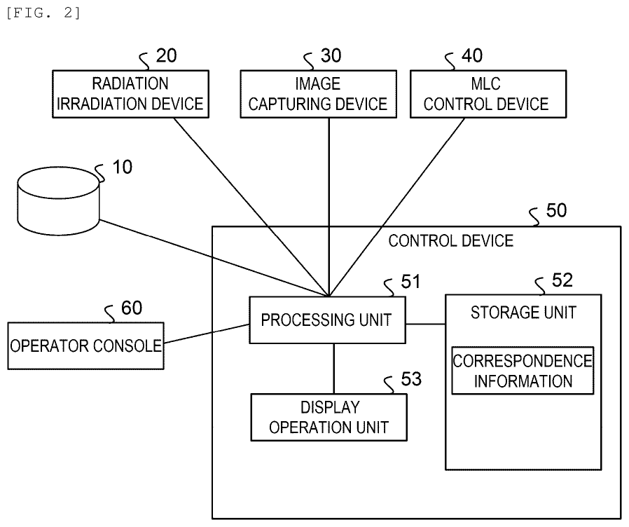

FIG. 2 is a diagram illustrating a control device 50 according to the embodiment. The control device 50 communicates with the database 10, the radiation irradiation device 20, the image capturing device 30, and the MLC control device 40. The database 10 stores patient information and treatment plans. The radiation irradiation device 20 includes a couch and a gantry, and irradiates a predetermined site of a patient lying on the couch with radiation for treatment. The image capturing device 30 captures an image of the patient lying on the couch. The image capturing device 30 is a device configured to image an affected part of the patient, such as a computed tomography (CT) device or a magnetic resonance imaging (MRI) device. The MLC control device 40 can change a shape of radiation emitted by the radiation irradiation device 20 according to a shape of a treatment target site by controlling each leaf of the MLC mounted on the radiation irradiation device 20. The radiation irradiation device 20 and the image capturing device 30 may be installed in a treatment room, and the radiation irradiation device 20 and the image capturing device 30 may be integrated.

The control device 50 is disposed, for example, in an operation room adjacent to the treatment room and is connected to an operator console 60 in the operation room. The operator console 60 includes various buttons and the like, and may be operated by a touch panel.

The control device 50 includes a processing unit 51, a storage unit 52, and a display operation unit 53 therein. The processing unit 51 is, for example, a central processing unit (CPU). The storage unit 52 is a storage device such as a hard disk drive. The display operation unit 53 is, for example, a touch panel display.

The storage unit 52 stores correspondence information indicating correspondence between an image (flow operation image) to be displayed in the display area of the display operation unit 53 when a transition is made to a predetermined step and the predetermined step.

The processing unit 51 switches the image in the display area based on the correspondence information when a transition is made to the predetermined step. Further, the processing unit 51 can display an image (confirmation image) different from the image (flow operation image) associated with the current step in the display area based on a designation operation from the operator.

In order to switch between the display of the flow operation image and the display of the confirmation image, the display operation unit 53 has a function of receiving a designation operation and a function of receiving an operation for returning to the display of the flow operation image associated with the current step.

The processing unit 51 generates, from an output of a device that performs an operation related to a predetermined step based on the correspondence information, a flow operation image to be displayed in the display area when a transition is made to the predetermined step.

When any of the plurality of devices is designated by the designation operation, the processing unit 51 displays, in the display area, a confirmation image generated from an output of the designated device.

The display operation unit 53 includes the first display area A1 and the second display area A2.

The processing unit 51 uses the first display area A1 for constant display of a predetermined type of information, and uses the second display area A2 for display of flow operation image and the confirmation image.

The predetermined type of information displayed in the display area A1 is information desired to be displayed in any step, such as the patient information or a gantry state. The information displayed in the display area A2 is information related to the current step. The information related to the current step includes a flow operation image to which the current step is shown to correspond according to the correspondence information and a confirmation image for which the operator performs a designation operation in the current step.

When a part of the flow operation image or the confirmation image requires an area wider than another flow operation image or confirmation image, a part of the information in the display area A1 may not be displayed temporarily, and the display area A2 may be enlarged accordingly. When the display area is enlarged for the image to be displayed in response to the operator's request, the operator can confirm the image displayed in more detail.

The display operation unit 53 provides a plurality of tab areas near the display area A2. The tab areas correspond to a plurality of targets that can be designated by a designation operation and flow operation images, and each tab area implements a function of receiving the designation operation and a function of returning to the flow operation image. Therefore, the operator can easily switch display content of the display area A2 by an operation of selecting the tab. Conventionally, the operator needs to move his/her eyes to display screens of the respective devices each time, but as described above, the operator can access the image (flow operation image or confirmation image) to be obtained as necessary by the operation in the same display area, and the operability in advancing the treatment step is improved.

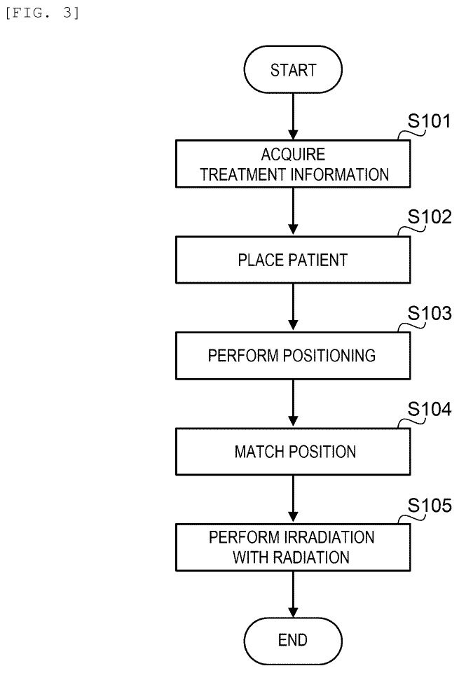

FIG. 3 is a flowchart illustrating processing operations of the control device 50. The processing operations in FIG. 3 correspond to a workflow of treatment steps of the radiation therapy, and sequentially transition from step S101 to step S105.

Step S101 is a treatment information acquisition step. In this step, the control device 50 acquires treatment information from the database 10. The treatment information includes patient information, a treatment plan, and the like.

Step S102 is a patient placement step. The operator places the patient on the couch in a predetermined posture. Thereafter, the operator moves from the treatment room to the operation room. The control device 50 locks a door of the treatment room, and executes subsequent steps on condition that the door is locked.

Step S103 is a positioning step. The control device 50 causes the image capturing device 30 to capture an image of the patient and compares the image with an image captured in advance. The image may be included in the treatment information acquired in step S101 or may be separately acquired. The control device 50 performs position adjustment of the couch and position adjustment of the gantry based on a comparison result of the images.

Step S104 is a position matching step. The control device 50 causes the image capturing device 30 to capture an image of the patient again, compares the image with the image captured in advance, and confirms whether the position is appropriately adjusted. If further adjustment is necessary, position adjustment of the couch and position adjustment of the gantry can be performed in the position matching step.

S105 is a radiation irradiation step. The control device 50 causes the MLC control device 40 to control each leaf of the MLC while causing the radiation irradiation device 20 to perform irradiation with radiation. For example, the gantry is rotated, and the irradiation is sequentially performed from different positions.

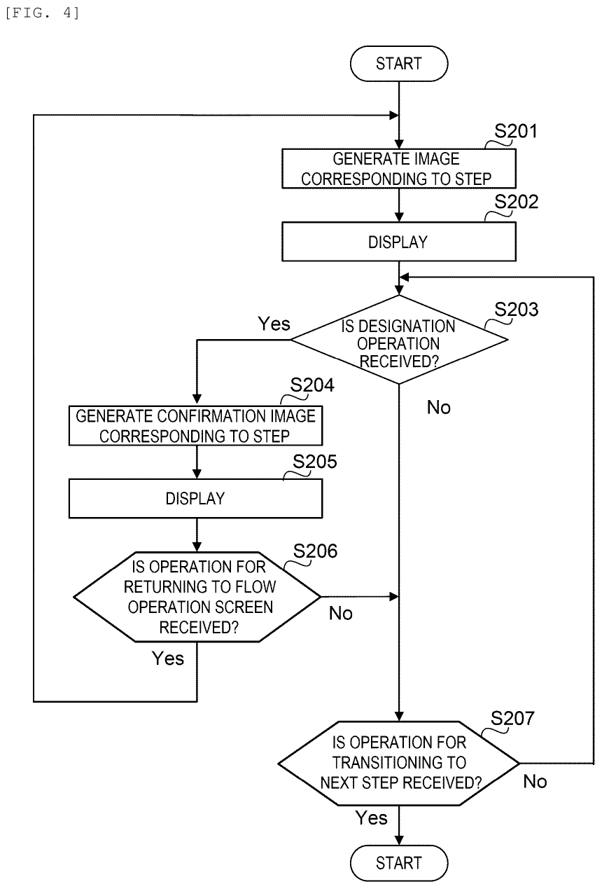

FIG. 4 is a flowchart illustrating display control in a step. When a certain step starts, the processing unit 51 of the control device 50 refers to the correspondence information in the storage unit 52 and generates a flow operation image corresponding to the current step (S201).

The processing unit 51 displays the generated flow operation image on the display operation unit 53 (S202). Thereafter, the processing unit 51 determines whether a designation operation for designating a target confirmation image is received by the display operation unit 53 (S203).

If the designation operation is received (S203; Yes), the processing unit 51 generates a confirmation image corresponding to the designation operation (S204). The processing unit 51 displays the generated confirmation image on the display operation unit 53 (S205). Thereafter, the processing unit 51 determines whether the display operation unit 53 receives an operation for returning to the flow operation image (S206).

If the operation for returning to the flow operation image is not received (S206; No), the process proceeds to S207. If the operation for returning to the flow operation image is received (S206; Yes), the process returns to step S201.

If the designation operation is not received in S203 (S203; No), the processing unit 51 proceeds to S207.

In S207, the processing unit 51 determines whether an operation for transitioning to the next step is received. If the operation for transitioning to the next step is received (S207; Yes), the flow operation image corresponding to the next step is generated. If the operation for transitioning to the next step is not received (step S207; No), the process returns to S203.

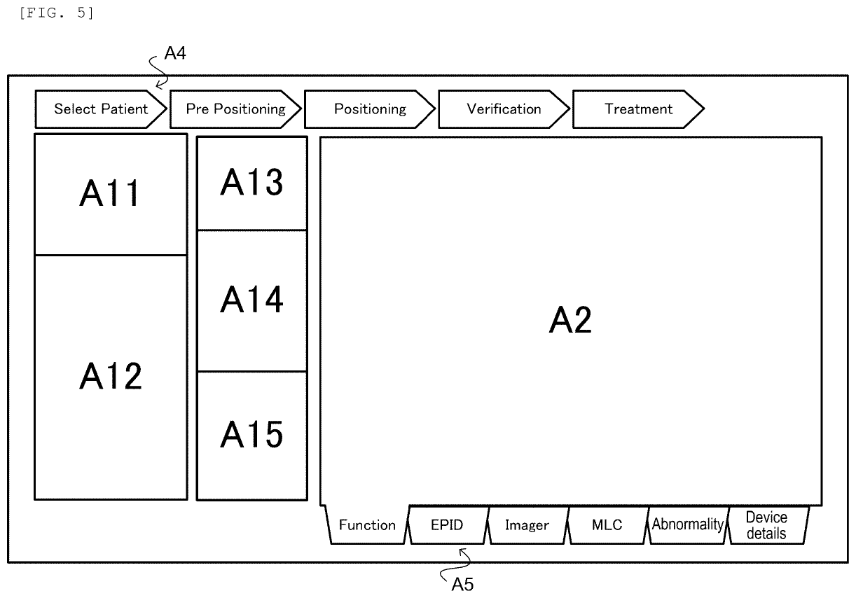

FIG. 5 is a diagram illustrating a screen of the display operation unit 53. The screen of the display operation unit 53 includes display areas A11 to A15 as the display area A1. The screen of the display operation unit 53 includes the display area A2 and a tab area A5. Further, the screen of the display operation unit 53 includes a progress status display A4 that occupies a progress status of the step.

The display area A11 displays, for example, a name, a gender, an age, and the like of the patient.

The display area A12 displays, for example, states of the couch and the gantry.

The display area A13 displays, for example, a treatment plan.

The display area A14 displays, for example, progress of radiation irradiation.

The display area A15 displays, for example, a control status of a multi-leaf collimator.

Among the display areas All to A15, the display areas A11 and A12 constantly display the same content in any step. Although the display areas A13 to A15 display the same content in any step, the display areas A13 to A15 are not displayed temporarily depending on display content in the display area A2, and the display area A2 is enlarged accordingly.

The progress status display A4 includes “Select Patient”, “Pre Positioning”, “Positioning”, “Verification”, and “Treatment”.

“Select Patient” corresponds to step S101 (treatment information acquisition step).

“Pre Positioning” corresponds to step S102 (patient placement step).

“Positioning” corresponds to step S103 (positioning step).

“Verification” corresponds to step S104 (position matching step).

“Treatment” corresponds to step S105 (radiation irradiation step).

The progress status of the workflow can be indicated by making a display mode of a status corresponding to the current step among these statuses different from display modes of other steps.

The display area A2 is an area for displaying the flow operation image and the confirmation screen. The tab area A5 is near the display area A2. The tab area A5 includes an “Abnormality” tab and a “Device details” tab in addition to tabs for displaying confirmation images such as a “Function” tab, an “EPID” tab, an “Imager” tab, and an “MLC” tab. The content displayed in the tab area A2 may be deleted, added, or changed depending on the operator.

The “Function” tab corresponds to the flow operation image. When the operator operates this tab during display of the confirmation image, the display area A2 returns to display of the flow operation image.

The “EPID” tab corresponds to an image acquired by the EPID of the radiation irradiation device 20. When the operator operates this tab, the display area A2 displays a confirmation image corresponding to an irradiation area acquired by the EPID.

The “Imager” tab corresponds to an image acquired by the image capturing device 30. When the operator operates this tab, the display area A2 displays a confirmation image that is a patient image acquired by the image capturing device 30.

The “MLC” tab corresponds to the MLC control device 40. When the operator operates this tab, the display area A2 displays a confirmation image of the MLC control device 40 related to an opening of the MLC.

The “Abnormality” tab relates to notification of an abnormality that occurs in the radiation therapy system. When the operator operates this tab, the display area A2 displays details about the abnormality that occurs. When an abnormality occurs, this tab may be automatically selected.

The “Device details” tab relates to a system configuration of the radiation therapy system. When the operator operates this tab, the display area A2 displays the system configuration of the radiation therapy system and displays a screen for receiving an input of a configuration change.

As described above, the disclosed control device 50 is a control device for a radiation therapy system in which steps of treatment transition in a workflow format, the control device including the storage unit 52, the processing unit 51, and the display operation unit 53 as an interface having one or more display areas. The storage unit 52 stores correspondence information between an image to be displayed in the display area when a transition is made to a predetermined step and the predetermined step. The processing unit 51 switches an image in the display area based on the correspondence information when a transition is made to the predetermined step. The processing unit 51 can display an image different from an image associated with the predetermined step in the display area based on a designation operation from an operator. The interface has a function of receiving the designation operation and a function of receiving an operation for returning to display of the image associated with the predetermined step.

Therefore, the operator can advance the treatment step by appropriately confirming necessary information, and the operability of the radiation therapy system is improved.

The disclosed control device 50 is connected to a plurality of devices related to the treatment. The processing unit 51 generates, from outputs of the plurality Of devices that perform operations related to a predetermined step, an image to be displayed in the display area when a transition is made to the predetermined step. When any of the plurality of devices is designated by the designation operation, the processing unit 51 displays an image generated from an output of the designated device in the display area.

As an example, the plurality of devices include a radiation irradiation device, an image capturing device, a multi-leaf collimator control device, and a treatment planning device.

Therefore, the operator can confirm the outputs of the plurality of devices related to the treatment in one display area.

The interface includes a first display area and a second display area. The processing unit uses the first display area for constant display of a predetermined type of information, and uses the second display area for display of an image associated with the step and display based on the designation operation.

As an example, the interface displays at least one of patient information and information indicating a state of a gantry in the first display area.

Therefore, the operator can commonly confirm some information in any step.

As an example, in a case of performing the display based on the designation operation, the processing unit can enlarge the second display area by not displaying a part of the first display area. When the display area is enlarged for the image to be displayed in response to the operator's request, the operator can confirm the image displayed in more detail.

The interface includes a plurality of tab areas, and one of the plurality of tab areas has a function of receiving an operation for returning to display of an image associated with the predetermined step, and the plurality of other tabs correspond to a plurality of devices respectively that are designable by the designation operation.

Therefore, the operator can easily switch the screen by selecting the tab.

The invention is not limited to the above-described embodiments, and includes various modifications. For example, the above-described embodiments £ have been described in detail to facilitate understanding of the invention, and the invention is not necessarily limited to those including all the configurations described above. In addition to deletion of a configuration, it is also possible to replace the configuration or add a configuration.

### REFERENCE SIGNS LIST

1: radiation therapy system
10: database
20: radiation irradiation device
30: image capturing device
40: MLC control device
50: control device
51: processing unit
52: storage unit
53: display operation unit
60: operator console
A1, A2: display area

1. A control device for a radiation therapy system in which steps of treatment transition in a workflow format, the control device comprising:
a storage unit;
a processing unit; and
an interface including one or more display areas, wherein
the storage unit stores correspondence information between an image to be displayed in the display area when a transition is made to a predetermined step and the predetermined step,
the processing unit switches an image in the display area based on the correspondence information when a transition is made to the predetermined step,
the processing unit is capable of displaying an image different from the image associated with the predetermined step in the display area, based on a designation operation from an operator, and
the interface has a function of receiving the designation operation and a function of receiving an operation for returning to display of the image associated with the predetermined step.

2. The control device for a radiation therapy system according to claim 1, wherein
the control device is connected to a plurality of devices related to the treatment,
the processing unit generates, from outputs of the plurality of devices that perform operations related to the predetermined step, the image to be displayed in the display area when a transition is made to the predetermined step, and
when any of the plurality of devices is designated by the designation operation, the processing unit displays an image generated from the output of the designated device in the display area.

3. The control device for a radiation therapy system according to claim 2, wherein
the plurality of devices include a radiation irradiation device, an image capturing device, a multi-leaf collimator control device, and a treatment planning device.

4. The control device for a radiation therapy system according to claim 1, wherein
the interface includes a first display area and a second display area, and
the processing unit uses the first display area for constant display of a predetermined type of information and uses the second display area for display of the image associated with the predetermined step and display based on the designation operation.

5. The control device for a radiation therapy system according to claim 4, wherein
the interface displays at least one of patient information and information indicating a state of a gantry in the first display area.

6. The control device for a radiation therapy system according to claim 4, wherein
the processing unit is capable of enlarging the second display area by not displaying a part of the first display area when performing the display based on the designation operation.

7. The control device for a radiation therapy system according to claim 2, wherein
the interface includes a plurality of tab areas, and one of the plurality of tab areas has a function of receiving the operation for returning to the display of the image associated with the predetermined step, and the plurality of other tabs correspond to a plurality of devices respectively that are designable by the designation operation.

8. A control method for a radiation therapy system in which steps of treatment transition in a workflow format, the control method to be executed by a control device including a storage unit, a processing unit, and an interface including one or more display areas, the control method comprising:
storing, by the storage unit, correspondence information between an image to be displayed in the display area when a transition is made to a predetermined step and the predetermined step;
switching, by the processing unit, an image in the display area based on the correspondence information when a transition is made to the predetermined step;
displaying, by the processing unit, an image different from an image associated with the predetermined step in the display area, based on a first operation from an operator received by the interface; and
returning, by the processing unit, to display of the image associated with the predetermined step, based on a second operation from the operator received by the interface.

9. A radiation therapy system comprising:
a radiation irradiation device;
an image capturing device;
a multi-leaf collimator control device;
a treatment planning device; and
a control device, wherein
the control device includes a storage unit, a processing unit, and an interface including one or more display areas, and causes steps of treatment to transition in a workflow format,
the storage unit stores correspondence information between an image to be displayed in the display area when a transition is made to a predetermined step and the predetermined step,
the processing unit switches an image in the display area based on the correspondence information when a transition is made to the predetermined step,
the processing unit is capable of displaying an image different from the image associated with the predetermined step in the display area based on a designation operation from an operator, and
the interface has a function of receiving the designation operation and a function of receiving an operation for returning to display of the image associated with the predetermined step.
